(19) United States
(12) Patent Application Publication
(10) Pub. No.: US 2016/0157830 A1
(43) Pub. Date: Jun. 9, 2016

(54) ULTRASONIC DIAGNOSTIC DEVICE AND ULTRASONIC IMAGE GENERATION METHOD

(71) Applicant: FUJIFILM Corporation, Tokyo (JP)

(72) Inventor: Kimito KATSUYAMA, Ashigara-kami-gun (JP)

(73) Assignee: FUJIFILM Corporation, Tokyo (JP)

(21) Appl. No.: 15/040,377

(22) Filed: Feb. 10, 2016

Related U.S. Application Data
(63) Continuation of application No. PCT/JP2014/060954, filed on Apr. 17, 2014.

(30) Foreign Application Priority Data
Aug. 27, 2013 (JP) 2013-175955

(51) Int. Cl. A61B 8/08; A61B 8/00

(52) U.S. Cl. CPC A61B 8/5207; A61B 8/4444; A61B 8/5238; A61B 8/4483; A61B 8/54

(57) ABSTRACT

An ultrasonic diagnostic device includes: a probe including a plurality of elements that generate and transmit ultrasonic waves and receive ultrasonic waves reflected from a subject; a transmission unit that transmits ultrasonic beams toward the subject from the plurality of elements of the probe; an image generation unit that generates an ultrasonic image by performing reception focusing for reception signals obtained by receiving the ultrasonic waves reflected from the subject in the plurality of elements of the probe; and a control unit that, when performing reception focusing in a direction different from a normal direction of each of the elements that form a reception opening of the probe, controls the image generation unit to generate an ultrasonic image in a direction different from the normal direction using only a signal having a predetermined low frequency band among the reception signals.

### CROSS-REFERENCE TO RELATED APPLICATIONS

This application is a Continuation of PCT International Application No. PCT/JP2014/060954 filed on Apr. 17, 2014, which claims priority under 35 U.S.C. §119(a) to Japanese Patent Application No. 2013-175955 filed on Aug. 27, 2013. The above application is hereby expressly incorporated by reference, in its entirety, into the present application.

### BACKGROUND OF THE INVENTION

1. Field of the Invention

The present invention relates to an ultrasonic diagnostic device and an ultrasonic image generation method, and in particular, to an ultrasonic diagnostic device and an ultrasonic image generation method for generating an ultrasonic image by receiving ultrasonic echoes, which are reflected from an oblique direction with respect to the normal direction of each element after transmitting ultrasonic beams from a plurality of elements of a probe.

2. Description of the Related Art

Conventionally, in the medical field, an ultrasonic diagnostic device using an ultrasonic image has been put into practical use. In general, this kind of ultrasonic diagnostic device generates an ultrasonic image by transmitting an ultrasonic beam toward a subject from the ultrasonic probe, receiving an ultrasonic echo from the subject using the ultrasonic probe, and electrically processing the reception signal.

In such an ultrasonic diagnostic device, a tomographic image of the inside of the subject located immediately below the probe can be observed in real time. Accordingly, for example, when inserting the needle to the target location in the subject, an ultrasonic image of the inside of the subject is generated by placing the probe immediately above a target location and the needle is obliquely inserted toward the target location from the vicinity of the probe, so that the needle is inserted while checking the position of the needle in the subject on an ultrasonic image.

However, in general, the surface of the needle is smooth. Accordingly, an ultrasonic beam having propagated through the subject from the probe is likely to be regularly reflected on the surface of the needle. In addition, since the needle is obliquely inserted toward the target location, it may be difficult to visualize the needle by capturing the specular reflection on the needle surface of the ultrasonic beam transmitted in the normal direction of the probe in the reception opening of the probe.

Therefore, visualizing the needle by transmitting an ultrasonic beam in a direction perpendicular to the needle instead of the normal direction of the probe and performing reception focusing has been known.

For example, JP2012-213606A discloses an ultrasonic diagnostic device that generates a first image by transmitting and receiving an ultrasonic beam in a first direction perpendicular to the element surface of a probe for the purpose of tissue imaging, generates a second image group by transmitting and receiving an ultrasonic beam in a plurality of second directions, which are different from the direction perpendicular to the element surface, for the purpose of needle imaging, generates an image in which a needle is visualized by analyzing the second image group, and combines the image with the first image.

According to the device disclosed in JP2012-213606A, since a direction perpendicular to the needle is included in the plurality of second directions, it is possible to generate an ultrasonic image in which the needle is satisfactorily visualized.

### SUMMARY OF THE INVENTION

However, each of a plurality of elements of the probe has an ultrasonic wave transmitting and receiving surface with a predetermined area. Accordingly, it is known that the strength of the ultrasonic wave transmitted and received in the deviated direction decreases in proportion to the amount of deviation from the normal direction, compared with the strength of the ultrasonic wave transmitted and received in the normal direction of the ultrasonic wave transmitting and receiving surface. That is, it is known that there is directivity.

For this reason, even if an ultrasonic image of the needle is generated by performing reception focusing in a direction deviated from the normal direction of the probe in a state in which the ultrasonic wave is perpendicular to the needle, both the strength of an ultrasonic wave transmitted from each element of the probe to the needle located in the direction and the strength of a signal obtained by receiving the reflected wave from the needle in each element are lower than the strength of the ultrasonic wave transmitted in the normal direction of the probe and the strength of a signal obtained by receiving the reflected wave from the normal direction. As a result, since the S/N ratio of the image is reduced, there has been a problem that it is difficult to visualize the needle clearly.

The present invention has been made in order to solve such a conventional problem, and it is an object of the present invention to provide an ultrasonic diagnostic device and an ultrasonic image generation method capable of generating a clear ultrasonic image even for a direction deviated from the normal direction of each element of the probe.

An ultrasonic diagnostic device according to the present invention includes: a probe including a plurality of elements that generate and transmit ultrasonic waves and receive ultrasonic waves reflected from a subject; a transmission unit that transmits ultrasonic beams toward the subject from the plurality of elements of the probe; an image generation unit that generates an ultrasonic image by performing reception focusing for reception signals obtained by receiving the ultrasonic waves reflected from the subject in the plurality of elements of the probe; and a control unit that, when performing reception focusing in a direction different from a normal direction of each of the elements that form a reception opening of the probe, controls the image generation unit to generate an ultrasonic image in a direction different from the normal direction using only a signal having a predetermined low frequency band among the reception signals.

The image generation unit can be configured to include: a first image generation section that generates an image signal along the normal direction by performing reception focusing in the normal direction of each of the elements, which form the reception opening of the probe, for the reception signals; and a second image generation section that generates an image signal in a direction different from the normal direction of each of the elements by performing reception focusing in a direction, which is different from the normal direction of each of the elements that form the reception opening of the probe, and using only the signal having the predetermined low frequency band for the reception signals.

The image generation unit can include a detection processing section that performs detection limited to the predetermined low frequency band.

It is preferable to further include an image combination unit that combines the image signal generated by the first image generation section and the image signal generated by the second image generation section.

When performing a sector scan for transmitting and receiving ultrasonic waves sequentially along a plurality of scanning lines with different directions from the plurality of elements of the probe, the control unit can be configured to control the image generation unit to generate an ultrasonic image by performing reception focusing in the direction of each scanning line and using only a signal having a lower frequency as an angle between the direction of each scanning line and the normal direction of each element that forms the reception opening of the probe becomes larger.

In this case, the image generation unit can include a detection processing section that performs detection limited to a low frequency band having a lower center frequency as the angle between the direction of each scanning line and the normal direction of each element that forms the reception opening of the probe becomes larger.

An ultrasonic image generation method according to the present invention is a method including: transmitting ultrasonic beams toward a subject from a plurality of elements of a probe; performing reception focusing in a direction, which is different from a normal direction of each of the elements that form a reception opening of the probe, for reception signals obtained by receiving ultrasonic waves reflected from the subject in the plurality of elements of the probe; and generating an ultrasonic image in a direction different from the normal direction using only a signal having a predetermined low frequency band among the reception signals.

According to the present invention, when performing reception focusing in a direction different from the normal direction of each of the elements that form the reception opening of the probe, the image generation unit is controlled so as to generate an ultrasonic image in a direction different from the normal direction using only the signal having a predetermined low frequency band among reception signals. Therefore, it becomes possible to generate a clear ultrasonic image even for a direction deviated from the normal direction of each element of the probe.

### BRIEF DESCRIPTION OF THE DRAWINGS

FIG. 1 is a block diagram showing the configuration of an ultrasonic diagnostic device according to a first embodiment of the present invention.

FIG. 2 is a diagram showing a state of transmission and reception of ultrasonic waves in the first embodiment.

FIG. 3 is a flowchart showing the operation in the first embodiment.

FIGS. 4A and 4B show ultrasonic images obtained by imaging an inserted needle, where FIG. 4A is an image obtained by performing reception focusing without limiting the frequency band and FIG. 4B is an image obtained by performing reception focusing by limiting the frequency band.

FIG. 5 is a block diagram showing the configuration of a needle image generation unit used in a modification example of the first embodiment.

FIG. 6 is a block diagram showing the configuration of a needle image generation unit used in another modification example of the first embodiment.

FIG. 7 is a block diagram showing the configuration of an ultrasonic diagnostic device according to a second embodiment.

FIG. 8 is a diagram showing a state of transmission and reception of ultrasonic waves in the second embodiment.

FIG. 9 is a flowchart showing the operation in the second embodiment.

### DESCRIPTION OF THE PREFERRED EMBODIMENTS

Hereinafter, embodiments of the present invention will be described with reference to the accompanying diagrams.

### First Embodiment

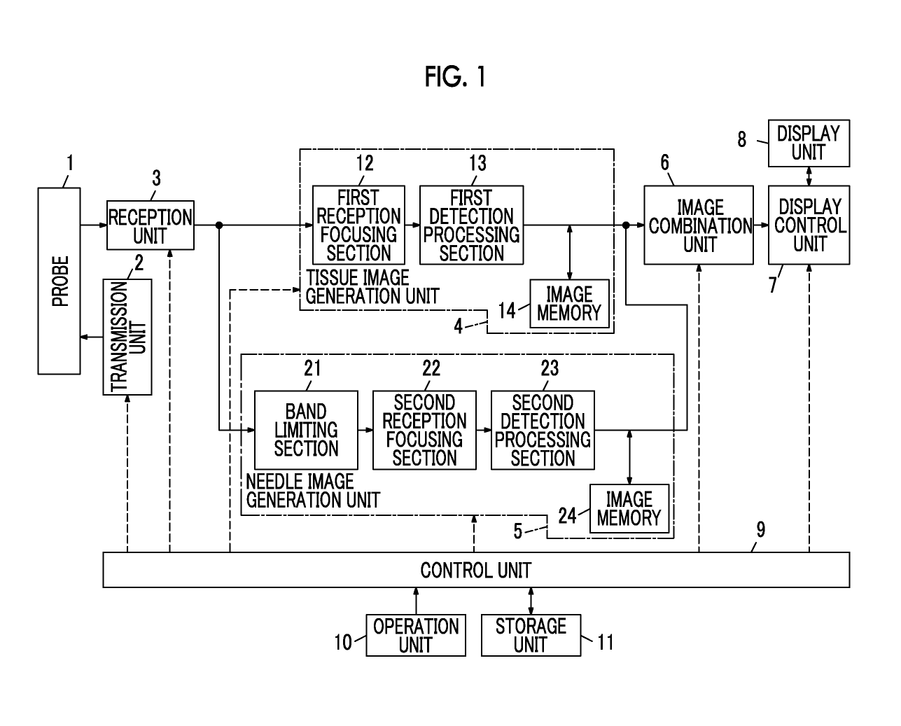

FIG. 1 shows the configuration of an ultrasonic diagnostic device according to a first embodiment of the present invention. The ultrasonic diagnostic device includes a probe 1, and a transmission unit 2 and a reception unit 3 are connected to the probe 1. A tissue image generation unit (first image generation unit) 4 and a needle image generation unit (second image generation unit) 5 are connected in parallel to the reception unit 3. An image combination unit 6 is connected to the tissue image generation unit 4 and the needle image generation unit 5, and a display unit 8 is connected to the image combination unit 6 through a display control unit 7.

A control unit 9 is connected to the transmission unit 2, the reception unit 3, the tissue image generation unit 4, the needle image generation unit 5, the image combination unit 6, and the display control unit 7, and an operation unit 10 and a storage unit 11 are connected to the control unit 9.

The tissue image generation unit 4 serves to generate a tissue image of the subject, that is located immediately below the probe 1, and includes a first reception focusing section 12 connected to the reception unit 3 and a first detection processing section 13 and an image memory 14 that are sequentially connected to the first reception focusing section 12. The first detection processing section 13 and the image memory 14 are connected to the image combination unit 6.

On the other band, the needle image generation unit 5 serves to generate an ultrasonic image of a needle inserted into the subject, and has the same configuration as the tissue image generation unit 4 except that a band limiting section 21 is provided. That is, the needle image generation unit 5 includes the band limiting section 21 connected to the reception unit 3 and a second reception focusing section 22, a second detection processing section 23, and an image memory 24 that are sequentially connected to the band limiting section 21. The second detection processing section 23 and the image memory 24 are connected to the image combination unit 6.

The probe 1 includes a plurality of elements arranged in a one-dimensional or two-dimensional manner. Each of these elements is an ultrasonic transducer, and transmits an ultrasonic wave according to the driving signal supplied from the transmission unit 2, receives an ultrasonic echo from the subject, and outputs a reception signal. For example, each ultrasonic transducer is formed by a transducer in which electrodes are formed at both ends of the piezoelectric body formed of piezoelectric ceramic represented by lead zirconate titanate (PZT), a polymer piezoelectric element represented by polyvinylidene fluoride (PVDF), piezoelectric single crystal represented by lead magnesium niobate-lead titanate solid solution (PMN-PT), or the like, and has an ultrasonic wave transmitting, and receiving surface with a predetermined area.

When a pulsed or continuous-wave voltage is applied to the electrodes of the transducer, the piezoelectric body expands and contracts to generate pulsed or continuous-wave ultrasonic waves from each transducer. By combination of these ultrasonic waves, an ultrasonic beam is formed. In addition, the respective transducers expand and contract by receiving the propagating ultrasonic waves, thereby generating electrical signals. These electrical signals are output as reception signals of the ultrasonic waves.

The transmission unit 2 includes a plurality of pulse generators, for example. Based on a transmission delay pattern selected according to the control signal from a control unit 9, the transmission unit 2 adjusts the amount of delay of each driving signal so that ultrasonic waves transmitted from the plurality of elements of the probe 1 form an ultrasonic beam, and supplies the adjusted signals to the plurality of elements.

The reception unit 3 amplifies the reception signal output from each element of the probe 1 and digitizes the amplified signal by A/D conversion.

The first reception focusing section 12 of the tissue image generation unit 4 generates delay correction data by performing delay correction for each reception signal amplified and digitized by the reception unit 3, adds up the pieces of delay correction data, and performs reception focusing processing. Through the reception focusing processing, a sound ray signal for tissue imaging with narrowed focus of the ultrasonic echo is generated.

The first detection processing section 13 generates a B-mode image signal for tissue imaging by correcting the attenuation due to the distance according to the depth of the reflection position of the ultrasonic wave for the sound ray signal generated by the first reception focusing section 12 and than performing envelope detection processing, and outputs the B-mode image signal to the image combination unit 6 or stores the B-mode image signal in the image memory 14.

The band limiting section 21 of the needle image generation unit 5 limits the reception signal amplified and digitized by the reception unit 3 to a signal having a predetermined low frequency band set in advance. That is, only a signal having a predetermined low frequency band, among the reception signals obtained by the reception unit 3, is extracted.

The second reception focusing section 22 generates delay correction data by performing delay correction for each reception signal limited to the signal having a predetermined low frequency band by the band limiting section 21, adds up the pieces of delay correction data, and performs reception focusing processing. Through the reception focusing processing, a sound ray signal for needle imaging with narrowed focus of the ultrasonic echo is generated.

The second detection processing section 23 generates a B-mode image signal for needle imaging by correcting the attenuation due to the distance according to the depth of the reflection position of the ultrasonic wave for the sound ray signal generated by the second reception focusing section 22 and then performing envelope detection processing, and outputs the B-mode image signal to the image combination unit 6 or stores the B-mode image signal in the image memory 24.

The image combination unit 6 converts (raster conversion) the B-mode image signal for tissue imaging output from the first detection processing section 13 of the tissue image generation unit 4 and the B-mode image signal for needle imaging output from the second detection processing section 23 of the needle image generation unit 5 into image signals according to the normal television signal scanning method and performs various kinds of required image processing, such as gradation processing, and then combines the B-mode image signal for tissue imaging and the B-mode image signal for needle imaging.

The display control unit 7 displays an ultrasonic image on the display unit 8 based on the B-mode image signal combined by the image combination unit 6.

The display unit 8 includes, for example, a display device, such as an LCD, and displays an ultrasonic image under the control of the display control unit 7.

The control unit 9 controls each unit of the ultrasonic diagnostic device based on the instruction input from the operation unit 10 by the operator.

The operation unit 10 is used when the operator performs an input operation, and can be formed by a keyboard, a mouse, a trackball, a touch panel, and the like.

The storage unit 11 stores an operation program and the like, and recording media, such as a hard disk, a flexible disk, an MO, an MT, a RAM, a CD-ROM, a DVD-ROM, an SD card, a CF card, and a USB memory, or a server may be used.

The first reception focusing section 12 and the first detection processing section 13 of the tissue image generation unit 4, the band limiting section 21, the second reception focusing section 22, and the second detection processing section 23 of the needle image generation unit 5, and the image combination unit 6 and the display control unit 7 are formed by using a CPU and an operation program causing the CPU to execute various kinds of processing. However, these may be formed by using digital circuits.

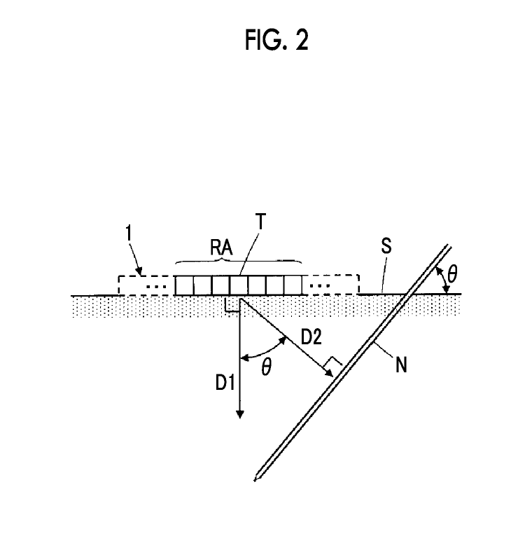

A method of transmitting and receiving an ultrasonic wave in the first embodiment will be described. As shown in FIG. 2, it is assumed that a needle N is inserted at an angle θ from the vicinity of the probe 1 in a state in which the probe 1 is in contact with the body surface of a subject S.

First, when imaging the tissue of the subject S located immediately below the probe 1, the transmission unit 2 transmits an ultrasonic beam in a normal direction D1 of each element from the probe 1. Then, the first reception focusing section 12 performs reception focusing in the normal direction D1 for reception signals obtained by the plurality of elements of the probe 1 that has received ultrasonic echoes, and the first detection processing section 13 performs detection.

On the other band, when imaging the needle N, the transmission unit 2 transmits an ultrasonic beam in a direction D2 perpendicular to the needle N from the probe 1. In this case, the direction D2 perpendicular to the needle N is expressed as a direction that is inclined by a puncture angle θ of the needle N from the normal direction D1. Then, the band limiting section 21 of the needle image generation unit 5 limits the reception signals obtained by the plurality of elements of the probe 1, which has received ultrasonic echoes, to signals in a predetermined low frequency band set in advance. Then, the second reception focusing section 22 performs reception focusing in the direction D2 perpendicular to the needle N, and the second detection processing section 23 performs detection.

The ultrasonic beam transmission direction and the direction of reception focusing do not necessarily need to be the direction D2 perpendicular to the needle N from the probe 1, and may be a direction toward the needle N rather than the normal direction D1, that is, a direction having an angle close to the right angle with respect to the needle N rather than the normal direction D1.

Here, assuming that the area of the ultrasonic wave transmitting and receiving surface of each element is the same, the directivity of the ultrasonic wave changes with the frequency of the ultrasonic wave. It is known that the directivity becomes higher as the frequency becomes higher and the directivity becomes lower as the frequency becomes lower. That is, when each element receives an ultrasonic echo signal, the ratio of the signal strength in a direction different from the normal direction of the ultrasonic wave transmitting and receiving surface to the signal strength in the normal direction becomes larger as the frequency of the ultrasonic wave becomes lower.

Therefore, when performing reception focusing in the direction D2 perpendicular to the needle N or in a direction toward the needle N rather than the normal direction D1, a clear ultrasonic image can be generated by imaging the needle N by limiting the reception signal to a signal having a predetermined low frequency band in order to increase the ratio of the signal strength in the direction D2 perpendicular to the needle N or in a direction toward the needle N rather than the normal direction D1 to the signal strength in the normal direction D1 of each element.

As shown in FIG. 2, in the case of a so-called linear type probe in which a plurality of elements are linearly arrayed, the normal directions D1 of the respective elements are parallel to each other. However, in a so-called convex type probe in which a plurality of elements are arrayed in a curved shape, the normal directions D1 of the respective elements are different. In this case, as shown in FIG. 2, reception focusing is performed in the direction D2 inclined by the puncture angle θ from the normal direction D1 of an element T located at the center among a plurality of elements that form a reception opening RA.

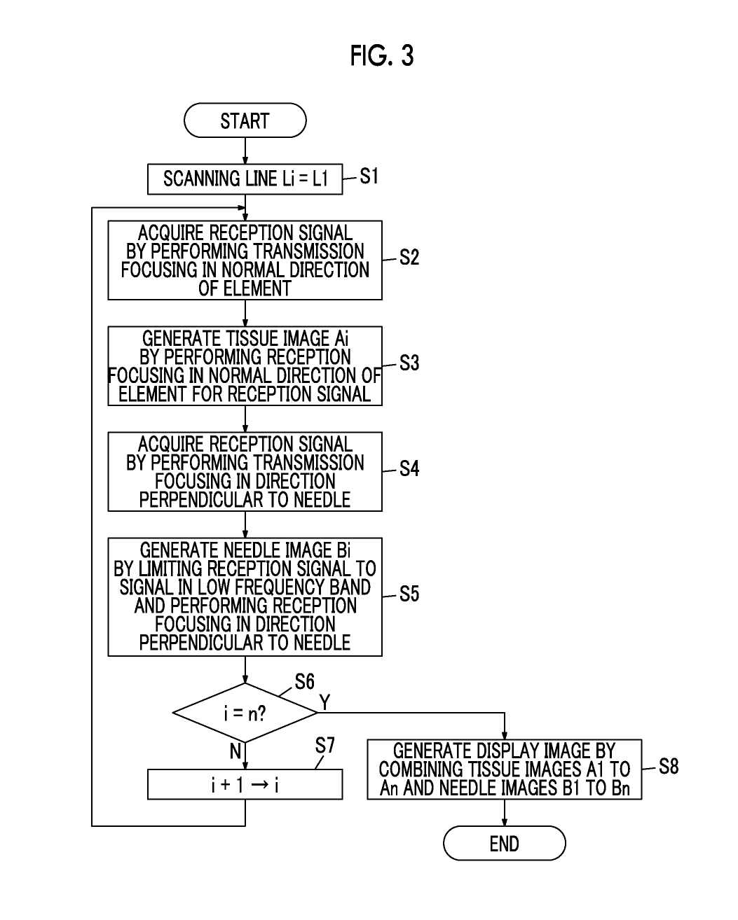

Next, an operation in the first embodiment will be described with reference to the flowchart shown in FIG. 3.

In the first embodiment, it is assumed that a tissue image in the normal direction D1 and a needle image in the direction D2 perpendicular to the needle N are generated by performing a scan by setting n scanning lines L1 to Ln in each of the normal direction D1 of each element of the probe 1 and the direction D2 perpendicular to the needle N.

First, in step S1, the scanning line L1 is initialized to L1. In step S2, a reception signal is acquired by performing transmission focusing in the normal direction of each element of the probe 1 corresponding to the first scanning line L1.

That is, according to the driving signal supplied from the transmission unit 2, transmission focusing is performed in the normal direction of each element from the plurality of elements that form a transmission opening corresponding to the scanning line L1, so that the ultrasonic beam is transmitted. Then, the reception unit 3 amplifies and digitizes the reception signal output from each element that has received the ultrasonic echo from the subject.

Then, in step S3, the reception signal is output to the tissue image generation unit 4 from the reception unit 3, and a tissue image A1 corresponding to the scanning line L1 is generated by performing reception focusing in the normal direction of each element for the reception signal.

That is, the first reception focusing section 12 generates delay correction data by performing delay correction for each reception signal so that reception focusing is performed in the normal direction of each element, and generates a sound ray signal for tissue imaging by adding up the pieces of delay correction data. The first detection processing section 13 performs envelope detection processing on the sound ray signal, thereby generating a B-mode image signal for tissue imaging. The B-mode image signal is stored in the image memory 14.

Then, in step S4, corresponding to the first scanning line L1, transmission focusing is performed in a direction perpendicular to the needle N from each element of the probe 1, thereby acquiring a reception signal.

That is, according to the driving signal supplied from the transmission unit 2, transmission focusing is performed in a direction perpendicular to the needle N from a plurality of elements that form a transmission opening corresponding to the scanning line L1, so that the ultrasonic beam is transmitted. The direction perpendicular to the needle N can be expressed as the direction D2 that is inclined by the puncture angle θ of the needle N from the normal direction D1 of each element, as shown in FIG. 2. For example, information regarding the puncture angle θ of the needle N input from the operation unit 10 by the operator is transmitted to the transmission unit 2 through the control unit 9, and the transmission unit 2 selects a transmission delay pattern corresponding to the direction D2 perpendicular to the needle N and performs transmission focusing.

Then, the reception unit 3 amplifies and digitizes the reception signal output from each element that has received the ultrasonic echo from the subject.

Then, in step S5, the reception signal is output to the needle image generation unit 5 from the reception unit 3, and is limited to a signal having a predetermined low frequency band. Then, a needle image B1 corresponding to the first scanning line L1 is generated by performing reception focusing in the direction D2 perpendicular to the needle N.

That is, the reception signal amplified and digitized by the reception unit 3 is limited to a signal having a predetermined low frequency band set in advance by the band limiting section 21 of the needle image generation unit 5. Then, the second reception focusing section 22 generates delay correction data by performing delay correction for each reception signal so that reception focusing is performed in the direction D2 perpendicular to the needle N, and generates a sound ray signal for needle imaging by adding up the pieces of delay correction data. The second detection processing section 23 performs envelope detection processing on the sound ray signal, thereby generating a B-mode image signal for needle imaging.

Only the signal having a predetermined low frequency band extracted by the band limiting section 21, among the reception signals obtained by the reception unit 3, is input to the second reception focusing section 22, and reception focusing is performed therefor. A sound ray signal for needle imaging generated by the reception focusing is input to the second detection processing section 23. Therefore, it is possible to generate a clear B-mode image signal in the direction D2 perpendicular to the needle N.

The B-mode image signal generated by the second detection processing section 23 is stored in the image memory 24.

Compared with the needle image generation unit 5, in the tissue image generation unit 4, the reception signal obtained by the reception unit 3 is input to the first reception focusing section 12 as it is without limiting the band of the reception signal, reception focusing is performed, and the sound ray signal for tissue imaging generated by the reception focusing is input to the first detection processing section 13. Therefore, the first detection processing section 13 performs detection for a wideband signal extending to a frequency band higher than the predetermined low frequency band in the needle image generation unit 5. Therefore, a B-mode image signal of a tissue image with excellent resolution is generated.

Thus, when the B-mode image signal of the tissue image A1 and the B-mode image signal of the needle image B1 corresponding to the first scanning line L1 are stored in the image memories 14 and 24, respectively, it is determined whether or not i=n, that is, it is determined whether or not the generation of tissue images and needle images has been completed for all of the n scanning lines L1 to Ln in step S6.

Here, since the value of i is still “1”, the process proceeds to step S7 to set the value of i to “2” by increasing the value of i by “1”, and then the process returns to step S2. Then, through steps S2 to S5, a B-mode image signal of a tissue image A2 and a B-mode image signal of a needle image B2 corresponding to the second scanning line L2 are generated, and are stored in the image memories 14 and 24, respectively.

Similarly, until i=n, the value of i is increased by 1 in a sequential manner, and steps S2 to S5 are repeated.

In this manner, when the generation of B-mode image signals of the tissue image and the needle image has been completed for all of the n scanning lines L1 to Ln, the process proceeds to step S8 from step S6. In step S8, B-mode image signals of tissue images A1 to An stored in the image memory 14 of the tissue image generation unit 4 and B-mode image signals of needle images B1 to Bn stored in the image memory 24 of the needle image generation unit 5 are raster-converted and are subjected to various kinds of image processing by the image combination unit 6. Then, obtained signals are combined to generate a B-mode image signal of a display image.

The B-mode image signal of the display image is output to the display control unit 7, and an ultrasonic image in which the tissue image and the needle image are combined is displayed on the display unit 8.

In addition, although the needle image is generated by setting the scanning lines in the direction D2 perpendicular to the needle N, it is also possible to generate a needle image by setting the scanning lines in a direction toward the needle N rather than the normal direction D1 of each element without being limited thereto.

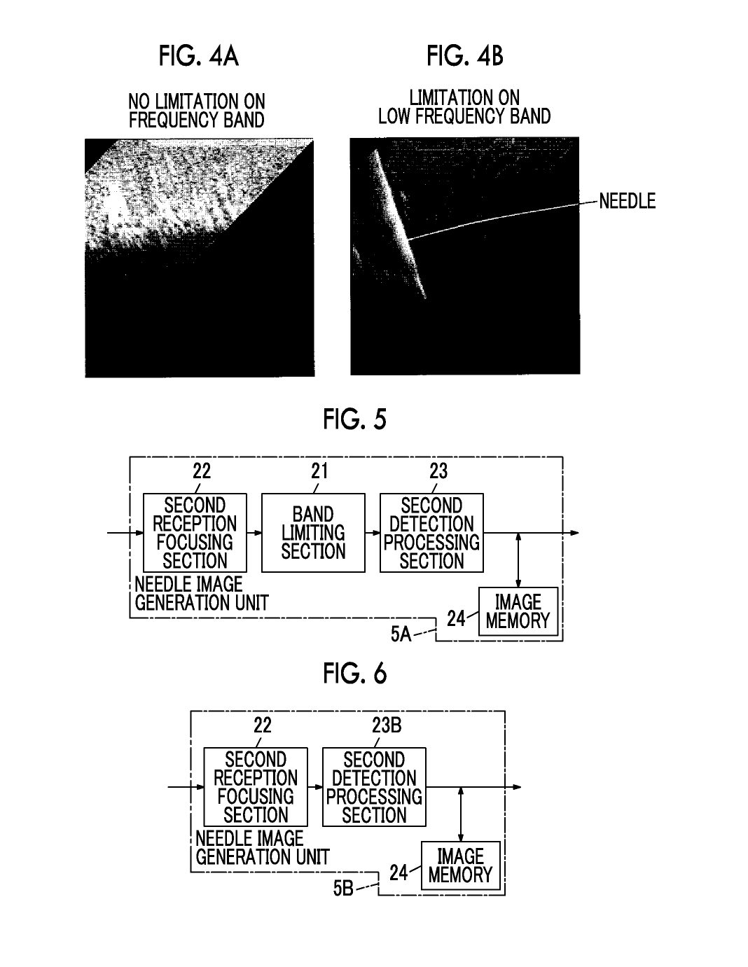

FIG. 4 shows a needle image obtained by imaging an inserted needle. FIG. 4A is an image generated by performing detection for a reception signal having a center frequency near 6 MHz without limiting the frequency using the band limiting section 21, and FIG. 4B is an image generated by performing detection by limiting a reception signal obtained by the reception unit 3 to a low frequency band equal to or lower than 3 MHz using the band limiting section 21. It can be seen that it is difficult to check the presence of the needle in the image shown in FIG. 4A, but the needle is visualized clearly in the image shown in FIG. 4B obtained by performing detection by limiting the signal to the low frequency band.

In the first embodiment described above, in step S4, a reception signal for needle images is acquired by performing transmission focusing in the direction D2 perpendicular to the needle N. However, the present invention is not limited thereto, and the reception signal acquired by performing transmission focusing in the normal direction D1 of each element in step S2 can be used for the generation of a needle image as well as the generation of a tissue image.

That is, for each scanning line Li, a needle image Bi may be generated by performing reception focusing in the direction D2 perpendicular to the needle N or in a direction toward the needle N for the reception signal acquired by performing transmission focusing in the normal direction D1 of each element.

In this case, since only one transmission is required for each scanning line Li, it is possible to improve the frame rate.

In this case, an ultrasonic beam extending radially in a direction toward the needle N as well as the normal direction D1 of each element may be transmitted from the probe 1, and an ultrasonic beam converging to the front of the transmission opening, that is, the subject, or an ultrasonic beam converging to the back of the transmission opening may be transmitted. It is also possible to transmit an ultrasonic beam of a plane wave.

In addition, a plurality of needle images Bi may be generated by performing reception focusing, for the same reception signal, in a plurality of different directions having larger angles with respect to the needle N than the normal direction D1, and an image in which the needle N is visualized most clearly may be selected from these. In this case, it is preferable to limit the reception signal to a signal having a low frequency band that is lower for a direction having a larger amount of deviation from the normal direction D1 of each element among the plurality of different directions.

In addition, in the first embodiment described above, as shown in FIG. 1, the band limiting section 21 of the needle image generation unit 5 is connected to the reception unit 3, and the reception signal obtained by the reception unit 3 is limited to a signal having a predetermined low frequency band. However, the present invention is not limited thereto, and the band limiting section 21 may be connected between the second reception focusing section 22 and the second detection processing section 23 as in a needle image generation unit 5A shown in FIG. 5. In this case, without limiting the frequency band of the reception signal obtained by the reception unit 3, the second reception focusing section 22 may generate a sound ray signal for needle imaging by performing reception focusing, and the band limiting section 21 limits the sound ray signal to a sound ray signal having a predetermined low frequency band. Also in this case, the second detection processing section 23 subsequently performs detection using only the signal having a predetermined low frequency band, thereby being able to generate a clear needle image.

In addition, as shown in FIG. 6, a needle image generation unit 5B not including the band limiting section 21 can be used, so that a second detection processing section 23B sets the reference frequency of detection to the center frequency of the predetermined low frequency band and adjusts the cutoff frequency. Thus, for the sound ray signal generated by the second reception focusing section 22, it is possible to perform detection using only the signal having the predetermined low frequency band.

By omitting steps S2 and S3 in FIG. 3, it is also possible to generate and display only a needle image without generating a tissue image.

In addition, the first embodiment is not limited to the case of clearly visualizing the needle N inserted into the subject, and can be widely applied to an object that is not easily visualized due to its specular reflection, such as the needle N. For example, even in the case of imaging bones, muscles, tendons, and the like in the body, it is possible to generate a clear ultrasonic image.

### Second Embodiment

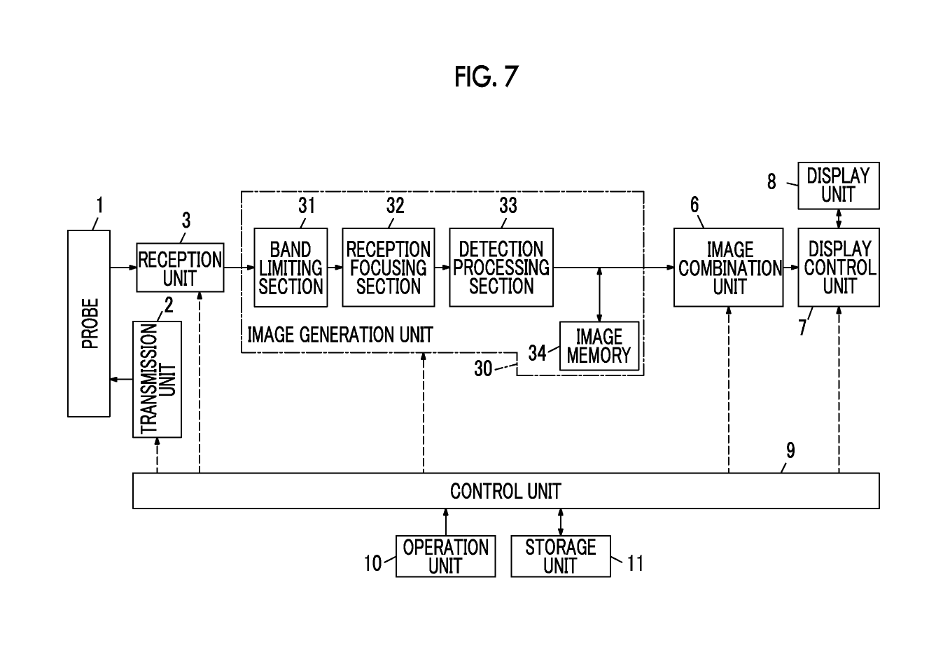

FIG. 7 shows the configuration of an ultrasonic diagnostic device according to a second embodiment. In particular, the ultrasonic diagnostic device is configured corresponding to a sector scan, and one image generation unit 30 is connected between the reception unit 3 and the image combination unit 6 instead of the tissue image generation unit 4 and the needle image generation unit 5 in the ultrasonic diagnostic device of the first embodiment shown in FIG. 1.

The image generation unit 30 includes a band limiting section 31 connected to the reception unit 3 and a reception focusing section 32, a detection processing section 33, and an image memory 34 that are sequentially connected to the band limiting section 31, and the detection processing section 33 and the image memory 34 are connected to the image combination unit 6.

Similar to the band limiting section 21 in the first embodiment, the band limiting section 31 limits the reception signal amplified and digitized by the reception unit 3 to a signal having a low frequency band. However, under the control of the control unit 9, corresponding to each scanning line of a sector scan, the band limiting section 31 limits the reception signal obtained by the reception unit 3 to a signal having a low frequency band having a lower center frequency as an angle between the direction of the scanning line and the normal direction of the element located at the center of the reception opening of the probe 1 becomes larger.

The reception focusing section 32 generates delay correction data by performing delay correction for each reception signal limited to the signal having a low frequency band by the band limiting section 31, adds up the pieces of delay correction data, and performs reception focusing processing. Through the reception focusing processing, a sound ray signal with narrowed focus of the ultrasonic echo is generated.

The detection processing section 33 generates a B-mode image signal corresponding to the scanning line by correcting the attenuation due to the distance according to the depth of the reflection position of the ultrasonic wave for the sound ray signal generated by the reception focusing section 32 and then performing envelope detection processing, and outputs the B-mode image signal to the image combination unit 6 or stores the B-mode image signal in the image memory 34.

The image combination unit 6 converts (raster conversion) the B-mode image signal corresponding to each scanning line into an image signal according to the normal television signal scanning method, and performs various kinds of required image processing, such is gradation processing, to generate a B-mode image signal of a display image.

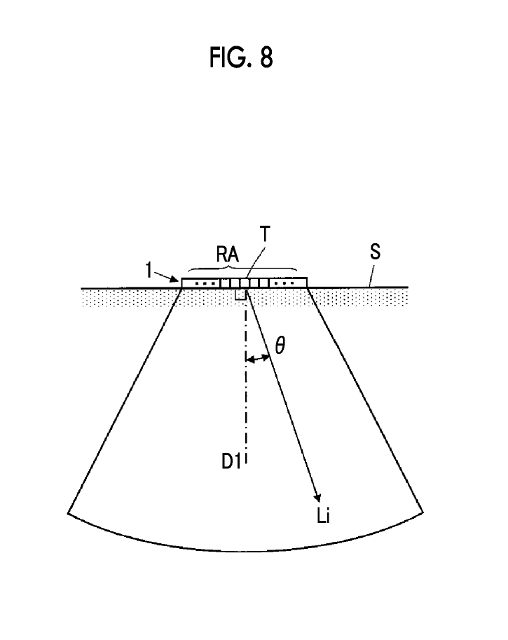

A method of transmitting and receiving an ultrasonic wave in the second embodiment will be described. As shown in FIG. 8, it is assumed that a sector scan is performed in a state in which the probe 1 is in contact with the body surface of the subject S. That is, transmission and reception of ultrasonic waves are sequentially performed along a plurality of scanning lines Li with different directions from a plurality of elements of the probe 1.

Assuming that the angle between the scanning line Li and the normal direction D1 of each element is θi, the transmission unit 2 transmits an ultrasonic beam in a direction along the scanning line Li, that is, in a direction of the angle θi with respect to the normal direction D1. Then, the reception focusing section 32 performs reception focusing in a direction of the scanning line Li for reception signals obtained by the plurality of elements of the probe 1 that has received ultrasonic echoes, and the band limiting section 31 limits the reception signals to signals in a low frequency band having a lower center frequency as the angle θi from the normal direction D1 becomes larger. Then, the detection processing section 33 performs detection.

Therefore, in a sector scan, it is possible to generate a clear ultrasonic image even in a direction deviated from the normal direction D1 or each element.

As shown in FIG. 8, in the case of a so-called linear type probe in which a plurality of elements are linearly arrayed, the normal directions D1 of the respective elements are parallel to each other. However, in a so-called convex type probe in which a plurality of elements are arrayed in a curved shape, the normal directions D1 of the respective elements are different. In this case, it is possible to perform detection for the signal having a low frequency band having a lower center frequency as the angle θi from the normal direction D1 of the element T located at the center, among a plurality of elements that form the reception opening RA, becomes larger.

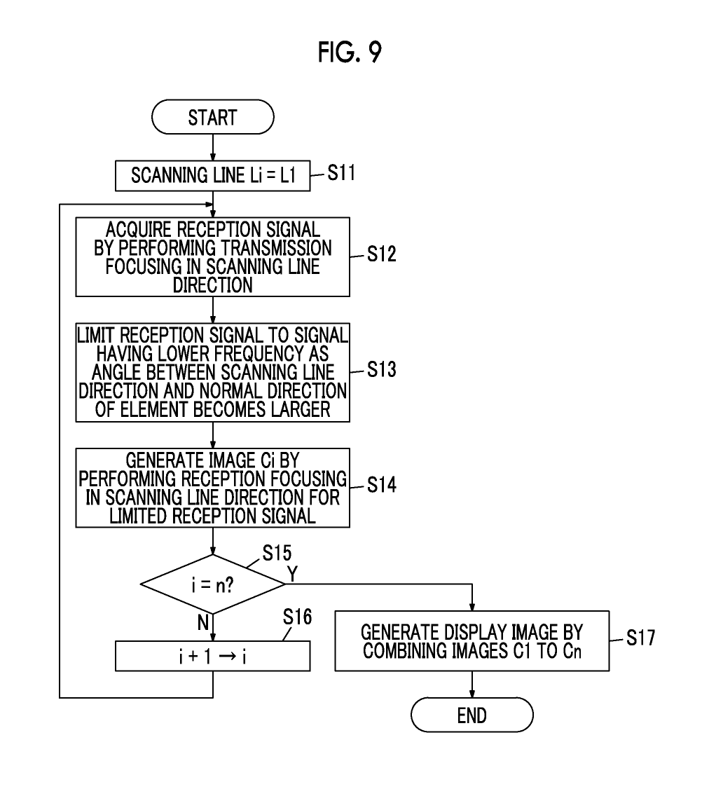

Next, an operation in the second embodiment will be described with reference to the flowchart shown in FIG. 9.

In the second embodiment, it is assumed that transmission and reception of ultrasonic waves are sequentially performed along n scanning lines L1 to Ln with different directions from a plurality of elements of the probe 1.

First, in step S11, the scanning line Li is initialized to L1. In step S12, a reception signal is acquired by performing transmission focusing in a direction along the first scanning line L1.

That is, according to the driving signal supplied from the transmission unit 2, transmission focusing is performed in a direction along the scanning line L1 from a plurality of elements that form a transmission opening corresponding to the first scanning line L1, so that the ultrasonic beam is transmitted. Then, the reception unit 3 amplifies and digitizes the reception signal output from each element that has received the ultrasonic echo from the subject.

Then, in step S13, the reception signal is output to the image generation unit 30 from the reception unit 3, and the band limiting section 31 limits the reception signal to a signal having a low frequency band having a low center frequency corresponding to the angle θ1 between the direction of the scanning line L1 and the normal direction D1 of each element.

Then, in step S14, the band limiting section 31 performs reception focusing in a direction along the scanning line L1 for the reception signal limited to the signal having a low frequency band, thereby generating a B-mode image signal of an image C1 corresponding to the first scanning line L1.

That is, the reception focusing section 32 generates delay correction data by performing delay correction for each reception signal limited to the signal having a low frequency band by the band limiting section 31 so that reception focusing is performed in a direction along the scanning line L1, and generates a sound ray signal corresponding to the first scanning line L1 by adding up the pieces of delay correction data. The detection processing section 33 performs detection for the sound ray signal.

Here, the reception signal obtained by the reception unit 3 is already limited to the signal having a low frequency band, which has a lower center frequency as the angle θi between the direction of the scanning line Li and the normal direction D1 of each element of the probe 1 becomes larger, according to each scanning line Li of the sector scan by the band limiting section 31. Therefore, even for the scanning line Li having a large angle θi from the normal direction D1 of each element of the probe 1, it is possible to generate a clear B-mode image signal.

The B-mode image signal generated by the detection processing section 33 is stored in the image memory 34.

Thus, when the image signal of the image C1 corresponding to the first scanning line L1 is stored in the image memory 34, it is determined whether or not i=n, that is, it is determined whether or not the generation of image signals has been completed for all of the n scanning lines Li in step S15.

Here, since the value of i is still “1”, the process proceeds to step S16 to set the value of i to “2” by increasing the value of i by “1”, and then the process returns to step S12. Then, through steps S12 to S14, a B-mode image signal of an image C2 corresponding to the second scanning line L2 is generated, and is stored in the image memory 34.

Similarly, until i=n, the value of i is increased by 1 in a sequential manner, and steps S12 to S14 are repeated.

When the generation of a B-mode image signal of an image Ci is completed for all of n scanning lines Li in this manner, the process proceeds to step S17 from step S15. In step S17, B-mode image signals of the images C1 to Cn stored in the image memory 34 are raster-converted by the image combination unit 6, and various kinds of image processing is performed to generate a B-mode image signal of a display image.

The B-mode image signal of the display image is output to the display control unit 7, and an ultrasonic image in which the images C1 to Cn of the n scanning lines L1 to Ln with different directions are combined is displayed on the display unit 8.

According to the second embodiment, also in the sector scan, it is possible to generate a clear ultrasonic image over the entire surface.

In addition, in the second embodiment described above, as shown in FIG. 7, the band limiting section 31 is connected to the reception unit 3, and the reception signal obtained by the reception unit 3 is limited to a signal having a low frequency band corresponding to the angle θi between the direction of the scanning line Li and the normal direction D1 of each element of the probe 1. However, the present invention is not limited thereto, and the band limiting section 31 may be connected between the reception focusing section 32 and the detection processing section 33. In this case, without limiting the frequency band of the reception signal obtained by the reception unit 3, the reception focusing section 32 may generate a sound ray signal by performing reception focusing, and the band limiting section 31 limits the sound ray signal to a sound ray signal having a low frequency band corresponding to the direction of the scanning line Li. Also in this case, the detection processing section 33 subsequently performs detection using only a signal having a lower frequency as the angle θi between the direction of the scanning line Li and the normal direction D1 of each element of the probe 1 becomes larger, thereby being able to generate a clear needle image.

In addition, the band limiting section 31 can be omitted. In this case, under the control of the control unit 9, the detection processing section 33 can perform detection using only a signal having a lower frequency as the angle θi between the direction of the scanning line Li and the normal direction D1 of each element of the probe 1 becomes larger, for the sound ray signal generated by the reception focusing section 32, by setting the reference frequency of detection as the center frequency of the low frequency band corresponding to the angle θi between the direction of the scanning line Li and the normal direction D1 of each element of the probe 1 and adjusting the cutoff frequency.

### EXPLANATION OF REFERENCES

1: probe

2: transmission unit

3: reception unit

4: tissue image generation unit

5, 5A, 5B: needle image generation unit

6: image combination unit

7: display control unit

8: display unit

9: control unit

10: operation unit

11: storage unit

12: first reception focusing section

13: first detection processing section

14, 24, 34: image memory

21, 31: band limiting section

22: second reception focusing section

23, 23B: second detection processing section

30: image generation unit

32: reception focusing section

33: detection processing section

D1: normal direction of element

D2: direction perpendicular to needle

RA: reception opening

T: element located at center

N: needle

θ: puncture angle

Li: scanning line

θi: angle of scanning line

S: subject

What is claimed is:

1. An ultrasonic diagnostic device, comprising:
a probe including a plurality of elements that generate and transmit ultrasonic waves and receive ultrasonic waves reflected from a subject;
a transmission unit that transmits ultrasonic beams toward the subject from the plurality of elements of the probe;
an image generation unit that generates an ultrasonic image by performing reception focusing for reception signals obtained by receiving the ultrasonic waves reflected from the subject in the plurality of elements of the probe; and
a control unit that, when performing reception focusing in a direction different from a normal direction of each of the elements that form a reception opening of the probe, controls the image generation unit to generate an ultrasonic image in a direction different from the normal direction using only a signal having a predetermined low frequency band among the reception signals.

2. The ultrasonic diagnostic device according to claim 1,
wherein the image generation unit includes a first image generation section that generates an image signal along the normal direction by performing reception focusing in the normal direction of each of the elements, which form the reception opening of the probe, for the reception signals; and
a second image generation section that generates an image signal in a direction different from the normal direction of each of the elements by performing reception focusing in a direction, which is different from the normal direction of each of the elements that form the reception opening of the probe, and using only the signal having the predetermined low frequency band for the reception signals.

3. The ultrasonic diagnostic device according to claim 1,
wherein the image generation unit includes a detection processing section that performs detection limited to the predetermined to frequency band.

4. The ultrasonic diagnostic device according to claim 2,
wherein the image generation unit includes a detection processing section that performs detection limited to the predetermined low frequency band.

5. The ultrasonic diagnostic device according to claim 2, further comprising:
an image combination unit that combines the image signal generated by the first image generation section and the image signal generated by the second image generation section.

6. The ultrasonic diagnostic device according to claim 4, further comprising:
an image combination unit that combines the image signal generated by the first image generation section and the image signal generated by the second image generation section.

7. The ultrasonic diagnostic device according to claim 1,
wherein, when performing a sector scan for transmitting and receiving ultrasonic waves sequentially along a plurality of scanning lines with different directions from the plurality of elements of the probe, the control unit controls the image generation unit to generate an ultrasonic image by performing reception focusing in the direction of each scanning line and using only a signal having a lower frequency as an angle between the direction of each scanning line and the normal direction of each element that forms the reception opening of the probe becomes larger.

8. The ultrasonic diagnostic device according to claim 7,
wherein the image generation unit includes a detection processing section that performs detection limited to a low frequency band having a lower center frequency as the angle between the direction of each scanning line and the normal direction of each element that forms the reception opening of the probe becomes larger.

9. An ultrasonic image generation method, comprising:
transmitting ultrasonic beams toward a subject from a plurality of elements of a probe;
performing reception focusing in a direction, which is different from a normal direction of each of the elements that form a reception opening of the probe, for reception signals obtained by receiving ultrasonic waves reflected from the subject in the plurality of elements of the probe; and
generating an ultrasonic image in a direction different from the normal direction using only a signal having a predetermined low frequency band among the reception signals.
